SEQUENCE LISTING (17 sequences)

SEQ ID NO: 1 - protein, 878 aa, Artificial Sequence
  Description of Artificial Sequence Synthetic polypeptide
MAKFLVIDGNSLLHRAYHAIPPLTTSGGLPTNAVYGFTNMLLRVITEEQPDMIAVAFDKGRITFRHDTFQEYKAQRPPMPDDLRPQLPILKDVLGALRISIHEAEGYEADDIIGTLVRVASENGHDSLILTGDKDILQLVGPHTQALLTRKGITELERYDMAKTRERFGITPAQLADLKGLVGDPSDNIPGVPGIGPKIAARLLADHGRLESVLENLDGLPPRVRQQLENFGDQARMSKKLATIDTGVPGLGKEDLKVWPGPDKPALLEIFNKLEFRSLVRRITEAPVTGGVPAGKPVEAFSPDYRLLDGPDHLETLLDQARRAGAVAVAYARGRSGIEALGFSVEAGNYLLPLGAADLEILAGVRRLFADAAVAKHMHNAKDFLRWAPDFDLANICFDSMVAAYLVNPLAANQQLEDVVHQYLNLVLVPDGPAAPATAADCIRRLYPVLRAELRGYELDYLFDRVELPLTRVLADMELAGVAVDRDQLEALSEEFGTRAGELAARICTLAGEDFNLNSPRQLGYILFEKLGLPAGKKTKTGYSTDAGVLLDLAEKHEIAALLLEYRQLIKLKTTYADGLAALVDPATGRLHTTLHQTVTNTGRLSSAEPNLQNIPIRMEEGRRIRKVFIPRDPARVLLTADYSQIELRILAHLSGDPALITAFRDEQDIHARTAAEVFGVPLEMVTPEMRSRAKAVNFGIIYGISDFGLARDLKVTRAEAREYIQRYFARLPGVKEYIDTAIRSARERGYVTTALNRRRPLPELFSANHTVRSFGERAAVNTPIQGTAADIIKLAMVKIHARMRERNLKTLMILQVHDELLFDVPAEEVCTVAPLVKAEMETVLPLNVPLTVDLKIGHNWYEVRKMDEVTQCLNFLK

SEQ ID NO: 2 - DNA, 2634 bp, Artificial Sequence
  Description of Artificial Sequence Synthetic polynucleotide
atggcaaaatttctggtgattgatggtaatagcctgctgcatcgtgcatatcatgcaattccgcctctgaccacaagcggtggtctgccgaccaatgcagtttatggttttaccaatatgctgctgcgtgtgattaccgaagaacagccggacatgattgcagttgcatttgataaaggtcgtatcacctttcgccatgataccttccaagaatataaagcacagcgtccgcctatgccggatgatctgcgtccgcagctgccgattctgaaagatgttctgggtgcactgcgtattagcattcatgaagccgaaggttatgaagcggatgatattattggcaccctggttcgtgttgccagcgaaaatggtcatgatagcctgattctgaccggtgataaagatattctgcagctggttggtccgcatacacaggcactgctgacccgtaaaggtattaccgaactggaacgttatgatatggcaaaaacccgtgaacgctttggtattacaccggcacagctggcagatctgaaaggtctggttggtgatccgagcgataatattccgggtgttcctggtattggtccgaaaattgcagcacgtctgctggccgatcatggtcgtctggaaagcgttctggaaaatctggatggtctgcctccgcgtgttcgtcagcagctggaaaattttggcgatcaggcacgtatgagcaaaaaactggcaaccattgataccggtgttccaggtctgggtaaagaagatttaaaagtttggcctggtccggataaacctgcactgctggaaattttcaacaaactggaatttcgtagcctggtgcgtcgtattacggaagcaccggttacaggtggtgttccggcaggtaaaccggttgaagcatttagtccggattatcgcctgctggatggaccggatcatctggaaacgctgctggatcaagcacgtcgtgccggtgcagttgccgttgcatatgcccgtggtcgtagcggtattgaagcactgggttttagcgttgaagcaggcaattatctgctgccgctgggtgcagcagatctggaaatcctggcaggcgttcgtcgtctgtttgcagatgcagccgttgcaaaacatatgcataatgccaaagattttctgcgttgggcacctgattttgatctggccaatatttgttttgatagcatggttgcagcctatctggttaatccgctggcagcaaatcagcaactggaagatgttgttcatcagtatctgaatctggttctggttccggatggtccggcagcaccggcaaccgcagcagattgtattcgtcgcctgtatccggttctgcgtgcagaactgcgtggttatgaactggattacctgtttgatcgtgttgaactgccgctgacacgtgttctggcagatatggaattagccggtgttgcagttgatcgtgatcagctggaagcactgagcgaagaatttggcacccgtgcgggtgaactggcagcacgtatttgtaccttagcgggtgaagatttcaatctgaatagtccgcgtcagctgggttatatcctgtttgaaaaactgggtctgcctgcaggtaaaaaaaccaaaaccggttatagcaccgatgccggtgttctgctggacctggcagaaaaacatgaaattgccgctctgctgctggaatatcgtcagctgattaaactgaaaaccacctatgcagatggcctggcagcgctggttgatccggcaacaggtcgtctgcataccacactgcatcagaccgttaccaataccggtcgtctgagcagtgccgaaccgaatctgcagaacattccgattcgtatggaagaaggtcgtcgtattcgtaaagtttttattccgcgtgatcctgcacgtgtgctgctgaccgcagattatagccagattgaactgcgcattctggcacatctgagtggcgatccagcactgattaccgcatttcgtgatgagcaggatattcatgcacgtaccgcagccgaagtttttggtgtgccgctggaaatggttacaccggaaatgcgtagccgtgcaaaagcagttaatttcggtattatctatggcatcagcgattttggactggcacgtgacctgaaagttacccgtgccgaagcacgtgaatatatccagcgttattttgcccgtctgcctggtgttaaagaatatattgatacagcaattcgtagcgcacgtgaacgcggttatgttaccaccgcactgaatcgtcgtcgtccgctgcctgaactgtttagcgcaaaccataccgttcgtagctttggtgaacgtgccgcagttaataccccgattcagggcaccgcagcggatatcattaaactggccatggttaaaattcatgcccgtatgcgtgaacgtaatctgaaaaccctgatgattctgcaggttcatgatgaactgctgtttgatgttcctgcagaagaagtttgtaccgttgcaccgctggttaaagcagaaatggaaacagttctgcctctgaatgttccgctgaccgttgatctgaaaattggccataattggtatgaggtgcgcaaaatggatgaagttacccagtgtctgaacttcctgaaa

SEQ ID NO: 3 - DNA, 42 bp, Artificial Sequence
  Description of Artificial Sequence Synthetic oligonucleotide
gcgcgtcccattcgccaatcgttttagagctgtgttgtttcg

SEQ ID NO: 4 - DNA, 30 bp, Artificial Sequence
  Description of Artificial Sequence Synthetic oligonucleotide
cgaaacaacacagctctaaaacgattggcg

SEQ ID NO: 5 - RNA, 42 nt, Artificial Sequence
  Description of Artificial Sequence Synthetic oligonucleotide
gcgcgucccauucgccaaucguuuuagagcuguguuguuucg

SEQ ID NO: 6 - DNA, 39 bp, Artificial Sequence
  Description of Artificial Sequence Synthetic oligonucleotide
gcctgcaggtcgactctagaggatccccgggtaccgagc

SEQ ID NO: 7 - DNA, 50 bp, Artificial Sequence
  Description of Artificial Sequence Synthetic oligonucleotide
  modified_base (35)..(38): a, c, t, g, unknown or other
  modified_base (41)..(43): a, c, t, g, unknown or other
  modified_base (45)..(49): a, c, t, g, unknown or other
gttttcgcatttatcgtgaaacgctttcgcgtttnnnntgnnncnnnnna

SEQ ID NO: 8 - DNA, 54 bp, Artificial Sequence
  Description of Artificial Sequence Synthetic oligonucleotide
  modified_base (6)..(10): a, c, t, g, unknown or other
  modified_base (12)..(14): a, c, t, g, unknown or other
  modified_base (17)..(20): a, c, t, g, unknown or other
ctagtnnnnngnnncannnnaaacgcgaaagcgtttcacgataaatgcgaaaac

SEQ ID NO: 9 - DNA, 67 bp, Artificial Sequence
  Description of Artificial Sequence Synthetic primer
aatgatacggcgaccaccgagatctacactatagcctatgcgacactcgtgaaacgctttcgcgttt

SEQ ID NO: 10 - DNA, 67 bp, Artificial Sequence
  Description of Artificial Sequence Synthetic primer
aatgatacggcgaccaccgagatctacacatagaggcatgcgacactcgtgaaacgctttcgcgttt

SEQ ID NO: 11 - DNA, 67 bp, Artificial Sequence
  Description of Artificial Sequence Synthetic primer
aatgatacggcgaccaccgagatctacaccctatcctatgcgacactcgtgaaacgctttcgcgttt

SEQ ID NO: 12 - DNA, 64 bp, Artificial Sequence
  Description of Artificial Sequence Synthetic primer
caagcagaagacggcatacgagatattactcgcgaggtcgagtgcatgaaacgctttcgcgttt

SEQ ID NO: 13 - DNA, 64 bp, Artificial Sequence
  Description of Artificial Sequence Synthetic primer
caagcagaagacggcatacgagattccggagacgaggtcgagtgcatgaaacgctttcgcgttt

SEQ ID NO: 14 - DNA, 64 bp, Artificial Sequence
  Description of Artificial Sequence Synthetic primer
caagcagaagacggcatacgagatcgctcattcgaggtcgagtgcatgaaacgctttcgcgttt

SEQ ID NO: 15 - DNA, 28 bp, Artificial Sequence
  Description of Artificial Sequence Synthetic primer
atgcgacactcgttcgtgcgtcagttca

SEQ ID NO: 16 - DNA, 31 bp, Artificial Sequence
  Description of Artificial Sequence Synthetic primer
cgaggtcgagtgcagttcgtgcgtcagttca

SEQ ID NO: 17 - DNA, 31 bp, Artificial Sequence
  Description of Artificial Sequence Synthetic primer
tgaactgacgcacgaactgcactcgacctcg

(19) United States
(12) Patent Application Publication
(10) Pub. No.: US 2021/0115414 A1
(43) Pub. Date: Apr. 22, 2021

(54) DNA POLYMERASES

(71) Applicants: THERMO FISHER SCIENTIFIC BALTICS UAB, Carlsbad, CA (US); BIGELOW LABORATORIES FOR OCEAN SCIENCES, Carlsbad, CA (US)

(72) Inventors: Ramunas STEPANAUSKAS, East Boothbay, ME (US); Gediminas ALZBUTAS, Vilnius (LT); Kotryna KAUNECKAITE-GRIGUOLE, Vilnius (LT)

(21) Appl. No.: 17/072,507

(22) Filed: Oct. 16, 2020

Related U.S. Application Data
(60) Provisional application No. 62/923,112, filed on Oct. 18, 2019.

(51) Int. Cl. C12N 9/12; C12N 15/10

(52) U.S. Cl. CPC C12N 9/1276; C12Y 207/07049; C12N 15/1096

(57) ABSTRACT

DNA polymerases with increased fidelity and/or reverse transcriptase activity are described for use in various applications. Compositions and kits comprising these DNA polymerases are also described. These DNA polymerases may be used in a variety of methods, including nucleic acid polynucleotide extension and amplification.

### CROSS-REFERENCE TO RELATED APPLICATION(S)

This application claims benefit of U.S. Application No. 62/923,112 filed Oct. 18, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 12, 2020 is named LT01436_SL.txt and is 15.892 bytes in size.

### FIELD

This application pertains to DNA polymerases with increased fidelity and/or reverse transcriptase activity, as well as use of such polymerases in various applications, including nucleic acid polynucleotide extension and amplification.

### BACKGROUND

Analysis of RNA and DNA molecules derived from various genes is very important in order to elucidate biological phenomena. In order to use some techniques to analyze nucleic acid molecules, the nucleic acid molecules may require amplification.

Nucleic acid amplification, often using thermophilic DNA polymerases, can permit rapid detection of a target nucleic acid sequence and/or provide sufficient quantities of a sample for further analysis or manipulation, such as sequencing, cloning, restriction digestion, hybridization, ligation, mutagenesis, recombination, etc. The accuracy of amplification can vary, however. DNA polymerases with strong proofreading activity (fidelity) increase the accuracy of DNA sequence replication. The ability of DNA polymerases to accurately replicate DNA sequences (i.e., attaining error-free sequences) significantly improves results in applications such as cloning, sequencing, and site-directed mutagenesis.

A DNA polymerase's fidelity is frequently expressed as the inverse of the error rate (fidelity=1/error rate); error rate refers to the number of misincorporated nucleotides per total number of nucleotides polymerized.

The error rate of Taq DNA polymerase, the most widely used polymerase in nucleic acid amplification, is about 2.2×10⁻⁵ errors per bp per duplication. Differences in assay methodology, reaction conditions, template sequences and error reporting units can yield different absolute values for error rates. For example, the reported error rate for Taq DNA polymerase, can range over 10-fold, from 1×10⁻⁵ to 2×10⁻⁴ errors/base/doubling (Cline J, Braman J C, Hogrefe H H. Nucleic Acids Research. 1996; 24(18):3546-51; Ling L L, Keohavong P, Dias C, Thilly W G. PCR Methods Appl. 1991; 1(1):63-9).

Often, the error rate of other DNA polymerases is expressed as relative to Taq DNA polymerase's fidelity. The fidelity of naturally occurring proofreading DNA polymerases, such as Pfu and KOD, is around 10 times that of Taq DNA polymerase. However, engineered DNA polymerases can have fidelity >50-100-times that of Taq DNA polymerase.

A number of different applications require high-fidelity DNA polymerases. Representative uses of high-fidelity DNA polymerases include (1) amplification of targeted genome regions using polymerase chain reaction (PCR) (see J. M. Bartlett and D. Stirling, Methods in Molecular Biology 226:3-6 (2003)) and similar techniques; (2) untargeted amplification of genomic DNA, such as rolling circle amplification (see F. B. Dean, et al. Genome Research 11:1095-1099 (2001)), multiple displacement amplification (see F. B. Dean et al., PNAS 99:5261-5266 (2002)), WGA-X (see R. Stepanauskas et al. Nature Communications 8:84 (2017)), multiple annealing and looping based amplification cycles (see C. Zong, et al. Science 338:1622-1626 (2012)), and others; (3) DNA sequencing that relies on these applications, including most current (“next-gen”) DNA sequencing technologies (e.g., Illumina, PacBio, Oxford Nanopores, SOLiD, Ion Torrent, Sanger, 454, etc.); and (4) genetic engineering applications that rely on these applications, e.g., those involving Clustered Regularly Interspaced Short Palindromic Repeats (CRISPR). Thus, new high-fidelity DNA polymerases may have use in a wide variety of applications.

The methods for analyzing mRNA molecules using a reverse transcriptase (RNA-dependent DNA polymerase) have now become indispensable experimental methods for studying genes. Furthermore, these methods, which have been applied to cloning techniques and PCR techniques, have become indispensable techniques not only for studying genes but also in wide variety of fields including biology, medicine, and agriculture.

The use of existing reverse transcriptases (RTs), most often from retroviral sources, may be inefficient for a variety of ways. Retroviral reverse transcriptases generally lack a proofreading 3′-5′ exonuclease domain, resulting in low-fidelity reverse transcription. Various efforts have been made to produce different enzyme compositions and methods for increasing the fidelity of polymerization on DNA or RNA templates. AccuScript (Agilent) is MMLV-RT derivative combined with 3′-5′ exonuclease, which is indicated by vendor to deliver 3.7-fold greater fidelity than other commercially available RTs. Transcriptor (Roche) is a blend of a recombinant reverse transcriptase and a proofreading enzyme, which is indicated by the vendor to have 7-fold higher fidelity compared to other commonly-used RTs.

Another factor influencing the efficiency of reverse transcription is the ability of RNA to form secondary structures. Such secondary structures can form, for example, when regions of RNA molecules have sufficient complementarity to hybridize and form double-stranded RNA. Generally, the formation of RNA secondary structures can be reduced by raising the temperature of solutions containing the RNA molecules. Thus, in many instances, users want to reverse transcribe RNA at temperatures above 37° C. However, many wild-type retroviral reverse transcriptases lose activity when incubated at temperatures much above 37° C. (e.g., 50° C.).

Previous research has shown that some thermophilic DNA polymerases (e.g. Tth, Taq, Bst DNA polymerase) could act as reverse transcriptases; however, for such activity some of these polymerases require additional Mn2+ cofactor ions, which are known to lower the accuracy of DNA polymerization. Others, if not requiring special cofactors, may have lower fidelity or lower thermophilic activity than that desired.

Thus as described above, there is continued need for new DNA polymerases with improved fidelity. There is also a need for new reverse transcriptases with improved fidelity and that would be active at elevated temperatures (i.e. thermophilic).

### SUMMARY

In accordance with the description, this application describes DNA polymerases. In some embodiments, these DNA polymerases may have polymerase activity and/or reverse transcriptase activity. In some embodiments, these DNA polymerases are thermophilic.

In some embodiments, a method of performing a DNA polymerization reaction is provided, the method comprising: a. providing a mixture in a buffer comprising (i) a template nucleic acid; (ii) at least one primer; (iii) nucleotides; and (iv) a DNA polymerase comprising (1) an amino acid sequence having at least 70%, 75%, 80%, 85%, 90%, 95%, 96%, 97%, 98%, or 99% identity to SEQ ID NO: 1, or (2) an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue; and b. incubating the mixture under conditions that: (i) permit hybridization of the at least one primer to the template nucleic acid; and (ii) permit extension of the at least one primer by polymerization of the nucleotides.

In some embodiments, a method of in vitro nucleic acid synthesis is provided, a method comprising contacting at least one primer and at least one template nucleic acid with a DNA polymerase in the presence of at least one dNTP in a buffer and incubating the reaction, wherein a DNA polymerase comprises: (1) an amino acid sequence having at least 70%, 75%, 80%, 85%, 90%, 95%, 96%, 97%, 98%, or 99% identity to SEQ ID NO: 1, or (2) an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue.

In some embodiments, the template nucleic acid used in the method of performing a DNA polymerization reaction or in vitro nucleic acid synthesis is RNA. In some embodiments the template nucleic acid is DNA.

In some embodiments, a method of reverse transcription and DNA amplification is provided, a method comprising contacting at least one primer and at least one template RNA with a DNA polymerase in the presence of at least one dNTP and incubating the reaction in a buffer under conditions for reverse transcription followed by conditions for DNA amplification, wherein a DNA polymerase comprises: (1) an amino acid sequence having at least 70%, 75%, 80%, 85%, 90%, 95%, 96%, 97%, 98%, or 99% identity to SEQ ID NO: 1, or (2) an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue.

In some embodiments, the DNA polymerase used in a method of performing a DNA polymerization reaction, in vitro nucleic acid synthesis or reverse transcription and DNA amplification DNA polymerase comprises SEQ ID NO: 1. In some embodiments, the DNA polymerase comprises amino acid sequence derived from SEQ ID NO: 1 by deletion of a consecutive amino acid sequence from the 1st to any amino acid up to 283rd amino acid residue. In some embodiments, the DNA polymerase comprises amino acid residues 284-878 of SEQ ID NO: 1. In some embodiments, the error rate of the DNA polymerase is at least 2-times lower than that of Taq DNA polymerase.

In some embodiments, the incubating in the method of performing a DNA polymerization reaction, in vitro nucleic acid synthesis or reverse transcription and DNA amplification is performed at temperature from 40° C. to 80° C. In some embodiments, the incubating is performed at 40° C. or higher. In some embodiments, the incubating is performed at 60° C. or higher.

This application also describes an isolated DNA polymerase comprising (1) an amino acid sequence having at least 70%, 75%, 80%, 85%, 90%, 95%, 96%, 97%, 98%, or 99% identity to SEQ ID NO: 1, or (2) an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue, is provided. In some embodiments, the DNA polymerase comprises SEQ ID NO: 1. In some embodiments, the DNA polymerase comprises amino acid sequence derived from SEQ ID NO: 1 by deletion of a consecutive amino acid sequence from the 1st to any amino acid up to 283rd amino acid residue. In some embodiments, the DNA polymerase comprises amino acid residues 284-878 of SEQ ID NO: 1. In some embodiments, the DNA polymerase has the error rate that is at least 2-times lower than that of Taq DNA polymerase under the same conditions. In some embodiments, a composition comprising the DNA polymerase and a storage solution is provided. In some embodiments, a composition comprises the DNA polymerase and a buffer.

This application also describes a kit comprising a DNA polymerase and at least one buffer suitable for use in a polymerase reaction.

In some embodiments, an isolated nucleic acid encodes a DNA polymerase described herein. In some embodiments, a vector comprises the nucleic acid. In some embodiments, a cell comprises the nucleic acid and/or the vector

This application also describes the use of a DNA polymerase as described herein in a buffer in in vitro nucleic acid synthesis. In some embodiments, the in vitro nucleic acid synthesis is for polymerase chain reaction, untargeted amplification of genomic DNA, DNA sequencing, or genetic engineering.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A and 1B provide data on DNA synthesis by Polymerase I (Pol I) using DNA as template. The 50 nM 6-fluorescein amidite (FAM)-labeled DNA-DNA oligoduplex (substrate) used for the experiment is shown in FIG. 1A. Reaction products analyzed by denaturing urea polyacrylamide gel electrophoresis are shown in FIG. 1B.

FIGS. 2A and 2B show DNA synthesis by Polymerase I using RNA as template (i.e., reverse transcriptase activity). The 6-fluorescein amidite (FAM)-labeled RNA-DNA oligoduplex (substrate) is shown in FIG. 2A. Reaction products analyzed by denaturing urea polyacrylamide gel electrophoresis are shown in FIG. 2B.

FIG. 3 shows Polymerase I activity using 6-fluorescein amidite (FAM)-labeled DNA-DNA oligoduplex (substrate) in different reaction buffers. Column numbers indicate the reaction buffer based on listing in Table 4, respectively. K is control with Taq DNA Polymerase in Buffer 1 from Table 4.

FIGS. 4A and 4B show Polymerase I activity in strand displacement amplification. M—O'GeneRuler 1 kb Plus DNA Ladder (Thermo Scientific), N—No template control samples, D—1 ng/L M13 ssDNA with annealed primer was used as a template.

### DESCRIPTION OF THE SEQUENCES

Table 1 provides a listing of certain sequences referenced herein.

TABLE 1
Description of the Sequences

| | | SEQ |
|---|---|---|
| | | ID |
| Description | Sequences | NO |
| Pol I amino | MAKFLVIDGNSLLHRAYHAIPPLTTSGGLPTNAVYGFTNMLLRVITEEQPDMIAVAFDKG | 1 |
| acid sequence | RITFRHDTFQEYKAQRPPMPDDLRPQLPILKDVLGALRISIHEAEGYEADDIIGTLVRVA | |
| | SENGHDSLILTGDKDILQLVGPHTQALLTRKGITELERYDMAKTRERFGITPAQLADLKG | |
| | LVGDPSDNIPGVPGIGPKIAARLLADHGRLESVLENLDGLPPRVRQQLENFGDQARMSKK | |
| | LATIDTGVPGLGKEDLKVWPGPDKPALLEIFNKLEFRSLVRRITEAPVTGGVPAGKPVEA | |
| | FSPDYRLLDGPDHLETLLDQARRAGAVAVAYARGRSGIEALGFSVEAGNYLLPLGAADLE | |
| | ILAGVRRLFADAAVAKHMHNAKDFLRWAPDFDLANICEDSMVAAYLVNPLAANQQLEDVV | |
| | HQYLNLVLVPDGPAAPATAADCIRRLYPVLRAELRGYELDYLFDRVELPLTRVLADMELA | |
| | GVAVDRDQLEALSEEFGTRAGELAARICTLAGEDFNLNSPRQLGYILFEKLGLPAGKKTK | |
| | TGYSTDAGVLLDLAEKHEIAALLLEYRQLIKLKTTYADGLAALVDPATGRLHTTLHQTVT | |
| | NTGRLSSAEPNLQNIPIRMEEGRRIRKVFIPRDPARVLLTADYSQIELRILAHLSGDPAL | |
| | ITAFRDEQDIHARTAAEVFGVPLEMVTPEMRSRAKAVNFGIIYGISDFGLARDLKVTRAE | |
| | AREYIQRYFARLPGVKEYIDTAIRSARERGYVTTALNRRRPLPELFSANHTVRSFGERAA | |
| | VNTPIQGTAADIIKLAMVKIHARMRERNLKTLMILQVHDELLFDVPAEEVCTVAPLVKAE | |
| | METVLPLNVPLTVDLKIGHNWYEVRKMDEVTQCLNFLK | |
| Pol I | ATGGCAAAATTTCTGGTGATTGATGGTAATAGCCTGCTGCATCGTGCATATCATGCAATT | 2 |
| nucleotide | CCGCCTCTGACCACAAGCGGTGGTCTGCCGACCAATGCAGTTTATGGTTTTACCAATATG | |
| sequence | CTGCTGCGTGTGATTACCGAAGAACAGCCGGACATGATTGCAGTTGCATTTGATAAAGGT | |
| | CGTATCACCTTTCGCCATGATACCTTCCAAGAATATAAAGCACAGCGTCCGCCTATGCCG | |
| | GATGATCTGCGTCCGCAGCTGCCGATTCTGAAAGATGTTCTGGGTGCACTGCGTATTAGC | |
| | ATTCATGAAGCCGAAGGTTATGAAGCGGATGATATTATTGGCACCCTGGTTCGTGTTGCC | |
| | AGCGAAAATGGTCATGATAGCCTGATTCTGACCGGTGATAAAGATATTCTGCAGCTGGTT | |
| | GGTCCGCATACACAGGCACTGCTGACCCGTAAAGGTATTACCGAACTGGAACGTTATGAT | |
| | ATGGCAAAAACCCGTGAACGCTTTGGTATTACACCGGCACAGCTGGCAGATCTGAAAGGT | |
| | CTGGTTGGTGATCCGAGCGATAATATTCCGGGTGTTCCTGGTATTGGTCCGAAAATTGCA | |
| | GCACGTCTGCTGGCCGATCATGGTCGTCTGGAAAGCGTTCTGGAAAATCTGGATGGTCTG | |
| | CCTCCGCGTGTTCGTCAGCAGCTGGAAAATTTTGGCGATCAGGCACGTATGAGCAAAAAA | |
| | CTGGCAACCATTGATACCGGTGTTCCAGGTCTGGGTAAAGAAGATTTAAAAGTTTGGCCT | |
| | GGTCCGGATAAACCTGCACTGCTGGAAATTTTCAACAAACTGGAATTTCGTAGCCTGGTG | |
| | CGTCGTATTACGGAAGCACCGGTTACAGGTGGTGTTCCGGCAGGTAAACCGGTTGAAGCA | |
| | TTTAGTCCGGATTATCGCCTGCTGGATGGACCGGATCATCTGGAAACGCTGCTGGATCAA | |
| | GCACGTCGTGCCGGTGCAGTTGCCGTTGCATATGCCCGTGGTCGTAGCGGTATTGAAGCA | |
| | CTGGGTTTTAGCGTTGAAGCAGGCAATTATCTGCTGCCGCTGGGTGCAGCAGATCTGGAA | |
| | ATCCTGGCAGGCGTTCGTCGTCTGTTTGCAGATGCAGCCGTTGCAAAACATATGCATAAT | |
| | GCCAAAGATTTTCTGCGTTGGGCACCTGATTTTGATCTGGCCAATATTTGTTTTGATAGC | |
| | ATGGTTGCAGCCTATCTGGTTAATCCGCTGGCAGCAAATCAGCAACTGGAAGATGTTGTT | |
| | CATCAGTATCTGAATCTGGTTCTGGTTCCGGATGGTCCGGCAGCACCGGCAACCGCAGCA | |
| | GATTGTATTCGTCGCCTGTATCCGGTTCTGCGTGCAGAACTGCGTGGTTATGAACTGGAT | |
| | TACCTGTTTGATCGTGTTGAACTGCCGCTGACACGTGTTCTGGCAGATATGGAATTAGCC | |
| | GGTGTTGCAGTTGATCGTGATCAGCTGGAAGCACTGAGCGAAGAATTTGGCACCCGTGCG | |
| | GGTGAACTGGCAGCACGTATTTGTACCTTAGCGGGTGAAGATTTCAATCTGAATAGTCCG | |
| | CGTCAGCTGGGTTATATCCTGTTTGAAAAACTGGGTCTGCCTGCAGGTAAAAAAACCAAA | |
| | ACCGGTTATAGCACCGATGCCGGTGTTCTGCTGGACCTGGCAGAAAAACATGAAATTGCC | |
| | GCTCTGCTGCTGGAATATCGTCAGCTGATTAAACTGAAAACCACCTATGCAGATGGCCTG | |
| | GCAGCGCTGGTTGATCCGGCAACAGGTCGTCTGCATACCACACTGCATCAGACCGTTACC | |
| | AATACCGGTCGTCTGAGCAGTGCCGAACCGAATCTGCAGAACATTCCGATTCGTATGGAA | |
| | GAAGGTCGTCGTATTCGTAAAGTTTTTATTCCGCGTGATCCTGCACGTGTGCTGCTGACC | |
| | GCAGATTATAGCCAGATTGAACTGCGCATTCTGGCACATCTGAGTGGCGATCCAGCACTG | |
| | ATTACCGCATTTCGTGATGAGCAGGATATTCATGCACGTACCGCAGCCGAAGTTTTTGGT | |
| | GTGCCGCTGGAAATGGTTACACCGGAAATGCGTAGCCGTGCAAAAGCAGTTAATTTCGGT | |
| | ATTATCTATGGCATCAGCGATTTTGGACTGGCACGTGACCTGAAAGTTACCCGTGCCGAA | |
| | GCACGTGAATATATCCAGCGTTATTTTGCCCGTCTGCCTGGTGTTAAAGAATATATTGAT | |
| | ACAGCAATTCGTAGCGCACGTGAACGCGGTTATGTTACCACCGCACTGAATCGTCGTCGT | |
| | CCGCTGCCTGAACTGTTTAGCGCAAACCATACCGTTCGTAGCTTTGGTGAACGTGCCGCA | |
| | GTTAATACCCCGATTCAGGGCACCGCAGCGGATATCATTAAACTGGCCATGGTTAAAATT | |
| | CATGCCCGTATGCGTGAACGTAATCTGAAAACCCTGATGATTCTGCAGGTTCATGATGAA | |
| | CTGCTGTTTGATGTTCCTGCAGAAGAAGTTTGTACCGTTGCACCGCTGGTTAAAGCAGAA | |
| | ATGGAAACAGTTCTGCCTCTGAATGTTCCGCTGACCGTTGATCTGAAAATTGGCCATAAT | |
| | TGGTATGAGGTGCGCAAAATGGATGAAGTTACCCAGTGTCTGAACTTCCTGAAA | |
| Bottom strand | GCGCGTCCCATTCGCCAATCGTTTTAGAGCTGTGTTGTTTCG | 3 |
| of DNA-DNA | | |
| oligoduplex, | | |
| 42 nt | | |
| Top strand of | CGAAACAACACAGCTCTAAAACGATTGGCG | 4 |
| oligoduplex, | | |
| 5′-FAM | | |
| labeled, 30 | | |
| nt | | |
| Bottom strand | GCGCGUCCCAUUCGCCAAUCGUUUUAGAGCUGUGUUGUUUCG | 5 |
| (RNA)of RNA- | | |
| DNA | | |
| oligoduplex, | | |
| 30 nt | | |
| M13_XbaI | GCCTGCAGGTCGACTCTAGAGGATCCCCGGGTACCGAGC | 6 |
| oligonucleotide | | |
| Transposon | GTTTTCGCATTTATCGTGAAACGCTTTCGCGTTTNNNNTGNNNCNNNNNA | 7 |
| end | | |
| containing 12 | | |
| randomized | | |
| nucleotides, | | |
| transferred | | |
| strand | | |
| Transposon | CTAGTNNNNNGNNNCANNNNAAACGCGAAAGCGTTTCACGATAAATGCGAAAAC | 8 |
| end | | |
| containing 12 | | |
| randomized | | |
| nucleotides, | | |
| non- | | |
| transferred | | |
| strand | | |
| Primer P5- | AATGATACGGCGACCACCGAGATCTACACTATAGCCTATGCGACACTCGTGAAACGCTTTC | 9 |
| D501 | GCGTTT | |
| Primer P5- | AATGATACGGCGACCACCGAGATCTACACATAGAGGCATGCGACACTCGTGAAACGCTTTC | 10 |
| D502 | GCGTTT | |
| Primer P5- | AATGATACGGCGACCACCGAGATCTACACCCTATCCTATGCGACACTCGTGAAACGCTTTC | 11 |
| D503 | GCGTTT | |
| Primer P7- | CAAGCAGAAGACGGCATACGAGATATTACTCGCGAGGTCGAGTGCATGAAACGCTTTCGCG | 12 |
| D701 | TTT | |
| Primer P7- | CAAGCAGAAGACGGCATACGAGATTCCGGAGACGAGGTCGAGTGCATGAAACGCTTTCGCG | 13 |
| D702 | TTT | |
| Primer P7- | CAAGCAGAAGACGGCATACGAGATCGCTCATTCGAGGTCGAGTGCATGAAACGCTTTCGCG | 14 |
| D703 | TTT | |
| Primer Read 1 | ATGCGACACTCGTTCGTGCGTCAGTTCA | 15 |
| Primer Read 2 | CGAGGTCGAGTGCAGTTCGTGCGTCAGTTCA | 16 |
| Primer Index | TGAACTGACGCACGAACTGCACTCGACCTCG | 17 |
| read | | |

### DESCRIPTION OF THE EMBODIMENTS

This application describes DNA polymerases. In some embodiments, these polymerases may be high-fidelity and/or thermophilic. In some embodiments, these enzymes have polymerase and/or reverse transcriptase, activity. DNA polymerases are used in a wide variety of methods; thus, polymerases with a unique profile may be useful for specific method. The DNA polymerase of the current disclosure does not have close homology with other DNA polymerases known in the art.

### I. Definitions

As used herein, “nucleic acid” refers to a covalently linked sequence of nucleotides or bases (e.g., ribonucleotides for RNA and deoxyribonucleotides for DNA). Nucleic acids include DNA and RNA.

As used herein, a “polymerase” catalyzes the polymerization of nucleotide (i.e., the polymerase activity). Generally, the enzyme will initiate synthesis at the 3′-end of the primer annealed to a polynucleotide template sequence and will proceed toward the 5′ end of the template strand. “DNA polymerase” catalyzes the polymerization of deoxynucleotides.

As used herein, a “reverse transcriptase” is an enzyme that generates DNA from an RNA template. Reverse transcription is the process of generating DNA from RNA.

As used herein, “3′ to 5′ exonuclease activity” is the ability of an enzyme to remove one or more nucleotides from the from the 3′ end of an oligonucleotide. The 3′ to 5′ exonuclease activity may be measured using any of the assays known in the art. 3′ to 5′ exonuclease activity of nucleic acid polymerases is often referred to as proofreading activity, as a DNA polymerase having this activity is capable of removing mismatched nucleotides from the 3′ terminus of a newly formed DNA strand.

The term “5′ to 3′ exonuclease activity” refers to the presence of an activity in a protein that is capable of removing nucleotides from the 5′ end of an oligonucleotide. 5′ to 3′ exonuclease activity may be measured using any of the assays known in the art.

The term “amplification”, as used herein, relates to the production of additional copies of a nucleic acid molecule. Amplification can be carried out using polymerase chain reaction (PCR) technologies or by other means including isothermal amplification methods such as, e.g., transcription mediated amplification, strand displacement amplification, rolling circle amplification, loop-mediated isothermal amplification.

The term “fidelity” as used herein refers to the accuracy of DNA polymerization by template-dependent DNA polymerase. The fidelity of a DNA polymerase is measured by the error rate (the frequency of incorporating an inaccurate nucleotide, i.e., a nucleotide that is not incorporated at a template-dependent manner). The accuracy or fidelity of DNA polymerization is maintained by both the polymerase activity and the 3′-5′ exonuclease activity of a DNA polymerase. Thus, a high-fidelity polymerase is a polymerase with a low error rate.

As used herein, the term “thermophilic” refers to an enzyme that is stable and capable to perform a reaction at elevated temperature, e.g. at temperatures of 45-80° C.

### II. DNA Polymerases

This application describes DNA polymerases. In some embodiments, these DNA polymerases have unique activity profiles. In some embodiments, these DNA polymerases are thermophilic. In some embodiments, these DNA polymerases have reverse transcriptase activity. In some embodiments, these DNA polymerases have high fidelity. In some embodiments, these DNA polymerases have strand-displacement activity.

In some embodiments, a DNA polymerase comprises an amino acid sequence having at least 70%, 75%, 80%, 85%, 90%, 95%, 96%, 97%, 98%, or 99% identity to SEQ ID NO: 1. In some embodiments, a DNA polymerase has at least 90%, 95%, 96%, 97%, 98%, or 99% identity to SEQ ID NO: 1. In some embodiments, a DNA polymerase comprises SEQ ID NO: 1. In some embodiments, variants of SEQ ID NO: 1 do not include the polymerase identical to SEQ ID NO: 1. In some embodiments, the DNA polymerase is an isolated DNA polymerase.

In some embodiments, a DNA polymerase comprises an amino acid sequence derived from amino acid sequence having at least 70%, 75%, 80%, 85%, 90%, 95%, 96%, 97%, 98%, or 99% identity to SEQ ID NO: 1. In some embodiments, a variant of DNA polymerase has polymerase activity and comprises an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd (and including 283rd amino acid residue) amino acid residue. The deletion is performed at the N-terminus of DNA polymerase and may inactivate 5′-exonuclease activity of DNA polymerase. In some embodiments, a variant DNA polymerase has improved strand-displacement activity compared to DNA polymerase without said deletion. In some embodiments, a DNA polymerase comprises an amino acid sequence derived from amino acid sequence having at least 70%, 75%, 80%, 85%, 90%, 95%, 96%, 97%, 98%, or 99% identity to SEQ ID NO: 1 and comprises amino acid residues 284-878 as compared to SEQ ID NO: 1. In some embodiments, a DNA polymerase comprises an amino acid sequence derived from SEQ ID NO: 1 and comprises amino acid residues 284-878 of SEQ ID NO: 1. In other embodiments, a DNA polymerase comprises an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to and including amino acid position 8, 19, 48, 58, 84, 111, 135, 160, 169 or 283. In some embodiments, a DNA polymerase comprises an amino acid sequence derived from SEQ ID NO: 1 by deletion of a consecutive amino acid sequence from the 1st to any amino acid up to and including amino acid position 8, 19, 48, 58, 84, 111, 135, 160, 169 or 283.

In some embodiments, the polymerases of the present disclosure can include without limitation polymerases and any subunits and truncations thereof, mutant polymerases, variant polymerases, recombinant, fusion or otherwise engineered polymerases, chemically modified polymerases, synthetic molecules or assemblies, and/or any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. In some embodiments, the modified polymerase can be a fusion protein comprising at least two portions linked to each other, where the first portion comprises a first polypeptide that can catalyze the polymerization of nucleotides into a nucleic acid strand, where the first portion is linked to a second portion that comprises a second polypeptide, such as, for example, a reporter enzyme, a processivity-enhancing domain, a tag polypeptide, or an affinity tag.

In some embodiments, DNA polymerase consists of an amino acid sequence derived from amino acid sequence having at least 70%, 75%, 80%, 85%, 90%, 95%, 96%, 97%, 98%, or 99% identity to SEQ ID NO: 1. In some embodiments, a variant of DNA polymerase has polymerase activity and consists of an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd (and including 283rd amino acid residue) amino acid residue. In some embodiments, a DNA polymerase consists of amino acid residues 284-878 of SEQ ID NO: 1.

In some embodiments, a DNA polymerase retains activity at a temperature of 40° C.-80° C. In some embodiments, a DNA polymerase retains activity at a temperature of 40° C. or higher. In some embodiments, a DNA polymerase retains activity at a temperature of 50° C., 51° C., 52° C., 53° C., 54° C., 55° C. or higher. In some embodiments, a DNA polymerase retains activity at a temperature of 60° C. or higher.

In some embodiments, a DNA polymerase retains reverse transcriptase activity at a temperature of 40° C.-60° C. In some embodiments, a DNA polymerase retains activity at a temperature of 40° C. or higher. In some embodiments, a DNA polymerase retains activity at a temperature of 50° C. or 55° C.

In some embodiments, an isolated nucleic acid encodes the DNA polymerase. In some embodiments, a vector comprises an isolated nucleic acid encoding the DNA polymerase. In some embodiments, a cell comprises a nucleic acid and/or vector of this application.

### III. Kits and Compositions

In some embodiments, a composition comprises a DNA polymerase and a suitable buffer. In some embodiments, a composition comprises a DNA polymerase and a storage solution.

In some embodiments, a kit comprises a DNA polymerase and at least one buffer suitable for a polymerase reaction. In some embodiments, the polymerase reaction is for a reverse transcription reaction.

A. Buffers

Buffers described in these kits or compositions may comprise a variety of components and/or additives. A “reaction buffer” refers to a buffer that is used for performing an enzymatic reaction with a DNA polymerase. A “component,” as used herein refers to a molecule normally included in a buffer. An “additive,” as used herein, refers to a molecule that improves a reaction efficiency (such as yield, fidelity, etc.) when using a buffer under certain conditions.

The DNA polymerase enzyme described herein can accept a wide variety of buffer conditions, showing distinct advantages in being able to function effectively in different environments. Thus, many different buffers and buffer ingredients may be used. This allows a user to choose a buffer of convenience or a buffer that benefits another step or element in a method comprising the reaction.

Certain buffer components and additives may improve activity under certain conditions. For example, certain components or additives of buffers may improve DNA polymerase activity at certain temperatures or with certain templates.

In some embodiments, the pH range of a reaction buffer is 7.5-8.8. In some embodiments, the pH of a reaction buffer is 7.5. In some embodiments, the pH of a reaction buffer is 7.9. In some embodiments, the pH of a reaction buffer is 8. In some embodiments, the pH of a reaction buffer is 8.8.

In some embodiments, the reaction buffer comprises mM Tris-HCl or Tris-Ac. In some embodiments, the reaction buffer comprises 10-75 mM Tris-HCl or Tris-Ac. In some embodiments, the reaction buffer comprises 10-75 mM Tris-HCl. In some embodiments, the reaction buffer comprises 10-75 mM Tris-Ac. In some embodiments, the reaction buffer comprises a mixture of both Tris-HCl and Tris-Ac, at the same or different concentrations.

In some embodiments, the reaction buffer comprises Tris-HCl. In some embodiments, the reaction buffer comprises 10 mM Tris-HCl. In some embodiments, the reaction buffer comprises 20 mM Tris-HCl. In some embodiments, the reaction buffer comprises 40 mM Tris-HCl. In some embodiments, the reaction buffer comprises 50 mM Tris-HCl. In some embodiments, the reaction buffer comprises 75 mM Tris-HCl.

In some embodiments, the reaction buffer comprises Tris-Ac. In some embodiments, the reaction buffer comprises 33 mM Tris-HCl.

In some embodiments, the reaction buffer comprises KCl and/or (NH₄)₂SO₄. In some embodiments, the reaction buffer comprises 0-66 mM KCl and/or (NH₄)₂SO₄.

In some embodiments, the reaction buffer comprises KCl. In some embodiments, the reaction buffer comprises 0-66 mM KCl. In some embodiments, the reaction buffer comprises 10 mM KCl. In some embodiments, the reaction buffer comprises 50 mM KCl. In some embodiments, the reaction buffer comprises 66 mM KCl. In some embodiments, the reaction buffer does not comprise KCl.

In some embodiments, the reaction buffer comprises CH3COOK. In some embodiments, the reaction buffer comprises 0-90 mM CH3COOK. In some embodiments, the reaction buffer comprises 10 mM CH3COOK. In some embodiments, the reaction buffer comprises 66 mM CH3COOK. In some embodiments, the reaction buffer comprises 90 mM CH3COOK. In some embodiments, the reaction buffer does not comprise CH3COOK.

In some embodiments, the reaction buffer comprises (NH₄)₂SO₄. In some embodiments, the reaction buffer comprises 0-66 mM (NH₄)₂SO₄. In some embodiments, the reaction buffer comprises 10 mM (NH₄)₂SO₄. In some embodiments, the reaction buffer comprises 20 mM (NH₄)₂SO₄. In some embodiments, the reaction buffer does not comprise (NH₄)₂SO₄.

In some embodiments, the reaction buffer comprises both KCl and (NH₄)₂SO₄, at the same or different concentrations.

In some embodiments, the reaction buffer comprises Mg²⁺. In some embodiments, the reaction buffer comprises 1.6-10 mM Mg²⁺. In some embodiments, the Mg²⁺ is supplied as a salt. In some embodiments, the salt is MgCl₂, MgSO₄, or Mg(Ac)₂.

In some embodiments, the reaction buffer comprises MgCl₂. In some embodiments, the reaction buffer comprises 1.6 mM MgCl₂. In some embodiments, the reaction buffer comprises 5 mM MgCl₂. In some embodiments, the reaction buffer comprises 10 mM MgCl₂.

In some embodiments, the reaction buffer comprises MgSO₄. In some embodiments, the reaction buffer comprises 2 mM MgSO₄.

In some embodiments, the reaction buffer comprises Mg(Ac)₂. In some embodiments, the reaction buffer comprises 10 mM Mg(Ac)₂.

In some embodiments, the reaction buffer comprises more than one Mg² salt. In some embodiments, the reaction buffer comprises more than one of MgCl₂, MgSO₄, or Mg(Ac)₂.

In some embodiments, the reaction buffer comprises bovine serum albumin (BSA). In some embodiments, the reaction buffer comprises 0.1 mg/mL BSA. In some embodiments, the reaction buffer does not comprise BSA.

In some embodiments, the reaction buffer comprises one or more deoxyribonucleotide triphosphate (dNTP). In some embodiments, the reaction buffer comprises 0.2-1.25 mM dNTP. In some embodiments, the reaction buffer comprises 0.2 mM dNTP. In some embodiments, the reaction buffer comprises 0.25 mM dNTP. In some embodiments, the reaction buffer comprises 0.625 mM dNTP. In some embodiments, the reaction buffer comprises 1.25 mM dNTP.

In some embodiments, the reaction buffer comprises one or more reducing agent. In some embodiments, the reducing agent is DTT. In some embodiments, the reaction buffer comprises 0-1 mM DTT. In some embodiments, the reaction buffer does not comprise DTT. In some embodiments, the reaction buffer does not comprise a reducing agent.

In some embodiments, the reaction buffer comprises one or more detergent. In some embodiments, the reaction buffer comprises 0.01-0.1% of one or more detergent. In some embodiments, the detergent is NP-40, Tween 20, and/or Triton X-100.

In some embodiments, the detergent is NP-40. In some embodiments, the reaction buffer comprises 0.01-0.1% NP-40. In some embodiments, the reaction buffer comprises 0.01% NP-40. In some embodiments, the reaction buffer comprises 0.08% NP-40. In some embodiments, the reaction buffer comprises 0.1% NP-40.

In some embodiments, the detergent is Tween 20. In some embodiments, the reaction buffer comprises 0.01-0.1% Tween 20. In some embodiments, the reaction buffer comprises 0.01% Tween 20. In some embodiments, the reaction buffer comprises 0.08% Tween 20. In some embodiments, the reaction buffer comprises 0.10% Tween 20.

In some embodiments, the detergent is Triton X-100. In some embodiments, the reaction buffer comprises 0.01-0.1% Triton X-100. In some embodiments, the reaction buffer comprises 0.01% Triton X-100. In some embodiments, the reaction buffer comprises 0.08% Triton X-100. In some embodiments, the reaction buffer comprises 0.1% Triton X-100.

In some embodiments, the reaction buffer comprises one detergent. In some embodiments, the reaction buffer comprises more than one detergent.

In some embodiments, the reaction buffer does not comprise a detergent.

Table 4 provides further examples of representative reaction compositions for primer elongation reactions. However, the reaction buffer of a kit or composition is not limited to the representative combinations listed in Table 4. In some embodiments, a reaction buffer excludes components or additives listed in Table 4. In some embodiments, a reaction buffer comprises additional components or additives than those listed in Table 4.

The DNA polymerase enzyme described herein can also accept a wide variety of buffer conditions when acting as reverse transcriptase. In some embodiments, the various reaction buffers, in particular as described above, are used. In further embodiments, the pH range of a reaction buffer is 7.5-9.0. In some embodiments, the pH of a reaction buffer is 9.0. In some embodiments, the reaction buffer comprises potassium acetate. In some embodiments, the reaction buffer comprises 90 mM of potassium acetate.

### IV. Methods of Use

The DNA polymerases described in this application can be used in a variety of methods.

In some embodiments, a method of performing a DNA polymerization reaction comprises providing a mixture comprising a template nucleic acid, at least one primer, nucleotides, and a DNA polymerase comprising an amino acid sequence having at least 70%, 75%, 80%, 85%, 90%, 95%, 96%, 97%, 98%, or 99% identity to SEQ ID NO: 1; and incubating the mixture under conditions that permit hybridization of the at least one primer to the template nucleic acid and permit extension of the at least one primer by polymerization of the nucleotides. In some embodiments, the polymerase comprises SEQ ID NO: 1. In some embodiments, variants of SEQ ID NO: 1 do not include the polymerase identical to SEQ ID NO: 1.

In some embodiments, a method of performing a DNA polymerization reaction comprises providing a mixture comprising a template nucleic acid, at least one primer, nucleotides, and a DNA polymerase comprising an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue. In some embodiments, DNA polymerase comprises amino acid sequence derived from SEQ ID NO: 1 by deletion of a consecutive amino acid sequence from the 1st to any amino acid up to 283rd amino acid residue. In some embodiment, DNA polymerase comprises amino acid residues 284-878 of SEQ ID NO: 1.

In some embodiments, a method of in vitro nucleic acid synthesis comprises contacting at least one primer and at least one template nucleic acid with a DNA polymerase comprising an amino acid sequence having at least 70%, 75%, 80%, 85%, 90%, 95%, 96%, 97%, 98%, or 99% identity to SEQ ID NO: 1 in the presence of at least one dNTP and incubating the reaction. In some embodiments, the polymerase comprises SEQ ID NO: 1. In some embodiments, variants of SEQ ID NO: 1 do not include the polymerase identical to SEQ ID NO: 1.

In some embodiments, a method of in vitro nucleic acid synthesis comprises contacting at least one primer and at least one template nucleic acid with a DNA polymerase comprising an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue. In some embodiments, DNA polymerase comprises amino acid sequence derived from SEQ ID NO: 1 by deletion of a consecutive amino acid sequence from the 1st to any amino acid up to 283rd amino acid residue. In some embodiment, DNA polymerase comprises amino acid residues 284-878 of SEQ ID NO: 1.

In some embodiments, a DNA polymerase comprising an amino acid sequence having at least 70%, 75%, 80%, 85%, 90%, 95%, 96%, 97%, 98%, or 99% identity to SEQ ID NO: 1 is used in in vitro nucleic acid synthesis. In some embodiments, the in vitro nucleic acid synthesis is for isothermal amplification, polymerase chain reaction, untargeted amplification of genomic DNA, DNA sequencing, or genetic engineering. In some embodiments, the DNA polymerase comprises SEQ ID NO: 1. In some embodiments, variants of SEQ ID NO: 1 do not include the polymerase identical to SEQ ID NO: 1.

In some embodiments, a DNA polymerase comprising an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue is used in in vitro nucleic acid synthesis. In some embodiments, DNA polymerase comprises amino acid sequence derived from SEQ ID NO: 1 by deletion of a consecutive amino acid sequence from the 1st to any amino acid up to 283rd amino acid residue. In some embodiment, DNA polymerase comprises amino acid residues 284-878 of SEQ ID NO: 1.

In some embodiments, the template nucleic acid is DNA or RNA. In some embodiments, the template nucleic acid is DNA. In some embodiments, the template nucleic acid is RNA.

In some embodiments, the DNA polymerase has high fidelity. In some embodiments, the error rate is at least 2-times lower than that of a different DNA polymerase. In some embodiments, the error rate is at least 2-times lower than that of Taq DNA polymerase under the same conditions. In some embodiments, the error rate is 2.5 times lower than that of Taq DNA polymerase. In some embodiments, the error rate is 3 times lower than that of Taq DNA polymerase. In some embodiments, the error rate is from 2 to 3 times lower than that of Taq DNA polymerase. In some embodiments, the error rate is more than 3 times lower than that of Taq DNA polymerase. Because error rate is inversely proportional to fidelity, a DNA polymerase with low error rate has high fidelity.

A. Reverse Transcriptase Activity

Enzymes with both DNA polymerase activity and reverse transcriptase activity can improve workflows in certain methods. For example, DNA polymerases with reverse transcriptase activity can enable both reverse transcription and amplification in one step for use in RNA detection from a sample in a single tube.

In some embodiments, the reverse transcriptase activity of the DNA polymerase is higher than some other DNA polymerases.

B. Fidelity

Improved fidelity (i.e., lower error rate) is critical to a wide range of applications to avoid introduction of mutations in nucleic acid sequences during extension or synthesis reactions.

In some embodiments, the error rate of the DNA polymerase is at least 2-times lower than that of a different DNA polymerase. In some embodiments, the error rate is at least 2-times lower than that of Taq DNA polymerase under the same conditions. In some embodiments, the error rate is about 2.1, about 2.2, about 2.3, about 2.4 or about 2.5 times lower than that of Taq DNA polymerase. In some embodiments, the error rate is 3 times lower than that of Taq DNA polymerase. In some embodiments, the error rate is from 2 to 3 times lower than that of Taq DNA polymerase. In some embodiments, the error rate is more than 3 times lower than that of Taq DNA polymerase.

A lower error rate indicates a higher fidelity of a DNA polymerase. In some embodiments, the error rate of a DNA polymerase described in this application is measured using the same method of fidelity determination as a comparator.

In some embodiments, the fidelity of the DNA polymerase is at least 2-times greater than that of a different DNA polymerase. In some embodiments, the fidelity is at least 2-times greater than that of Taq DNA polymerase under the same conditions. In some embodiments, the fidelity is about 2.1, about 2.2, about 2.3, about 2.4 or about 2.5 times higher than that of Taq DNA polymerase. In some embodiments, the fidelity is 3 times higher than that of Taq DNA polymerase. In some embodiments, the fidelity is from 2 to 3 times higher than that of Taq DNA polymerase. In some embodiments, the fidelity is more than 3 times higher than that of Taq DNA polymerase.

In some embodiments, the fidelity of a DNA polymerase described in this application is measured using the same method of fidelity determination as a comparator.

Examples 8 and 9 of the present application describe some means to measure error rates of a DNA polymerase. However, one skilled in the art could use any standard means to measure error rate and to determine fidelity.

C. Thermophilic Activity

Thermophilic activity refers to an enzyme that can perform a reaction at a high relative temperature. In other words, a thermophilic DNA polymerase can perform a reaction (i.e., polymerize DNA molecules) at a temperature at which some other DNA polymerases would not perform a reaction. Thermophilic DNA polymerases can be used in certain methods where a non-thermophilic DNA polymerase cannot be used.

In some embodiments, incubating of a reaction is performed at a temperature of 40° C.-80° C. In some embodiments, incubating of a reaction is performed at a temperature 40° C. or higher. In some embodiments, incubating of a reaction is performed at a temperature of 50° C., 51° C., 52° C., 53° C., 54° C., 55° C. or higher. In some embodiments, incubating of a reaction is performed at a temperature of 60° C. or higher.

In some embodiments, incubating of a reverse transcription reaction is performed at a temperature of 40° C.-60° C. In some embodiments, such reaction is performed at a temperature of 50° C. or 55° C.

### EXAMPLES

### Example 1. Cloning of Polymerase I Gene

Plasmid DNA containing the Polymerase I (Pol I) gene was synthesized by Invitrogen™ GeneArt. The nucleotide sequence SEQ ID NO: 2 denotes the gene sequence of Polymerase I, which encodes the amino acid sequence of Polymerase I (SEQ ID NO: 1).

Insert DNA containing the polymerase was amplified from plasmid DNAs using Invitrogen™ Platinum™ SuperFi™ Green DNA Polymerase (#10488-085). Amplified DNA fragments were extracted from agarose gel using Thermo Scientific™ GeneJET™ Gel Extraction and DNA Cleanup Micro Kit (#K0831). Ligation independent cloning of the polymerase was performed using Thermo Scientific™ aLICator™ LIC Cloning Set1 (#K1271).

The plasmid construct was transferred into E. coli strain DH10B using calcium chloride transformation method (see M. Dagert and S. Ehrlich Gene 6(1):23-8 (1979)). Colony PCR was performed with Thermo Scientific™ DreamTaq™ Green DNA Polymerase (#EP0711) to screen for plasmids carrying the desired genes. Successful transformants were confirmed by Sanger sequencing of the plasmids. The plasmid DNA was isolated from overnight bacterial culture using Thermo Scientific™ GeneJET™ Plasmid Miniprep Kit (#K0502).

### Example 2. Expression of Polymerase I

The plasmid construct of Example 1 was transferred into E. coli protein expression strain ER2566 which contains a gene of T7 RNA polymerase, using the calcium chloride transformation method. A colony of a transformant was transferred to 50 mL of LB media with ampicillin (100 μg/mL) and incubated with shaking at 37° C. overnight. Eight mL of starter cultures were transferred to 500 mL of LB media with ampicillin (100 μg/mL) and incubated with shaking at 37° C. for 3-4 hours until each culture's optical densities (OD600) reached 0.5-0.8. The media were cooled down to 16° C., and protein expression was induced by adding IPTG to a final concentration of 2 mM and then incubating at 16° C. with shaking overnight. Cells were collected by centrifuging at 20,000×g for 15 min at 4° C. The supernatant was removed, and cell pellet was frozen at −20° C. for later processing.

Two grams of the biomass was resuspended in 8.4 mL of buffer A (20 mM Tris-HCl pH 8, 50 mM NaCl, 2 mM EDTA, 0.5% Elugent™, 1 mM DTT) and then 80 μL of 100× Halt™ Protease Inhibitor Cocktail were added. Cell lysis was performed by sonication (Vibra-Cell™ Ultrasonic Liquid Processor) on ice using pulse mode (10 seconds on, 10 seconds off) and 50% amplitude for 7.5 minutes. Cell debris were pelleted by centrifuging at 30,000×g for 30 minutes at 4° C. Supernatant was filtered through 0.2 μm and was collected and stored at 4° C. for later processing.

### Example 3. Purification of Polymerase I

Polymerase I was further purified using 24 ml volume column with POROS HQ 50 resin (Applied Biosystems). The column was equilibrated with 5 resin-bed volumes of buffer A (20 mM Tris-HCl pH 8, 50 mM NaCl, 2 mM EDTA, 0.5% Elugent™), and then protein extracts were added to the resins. The resins were washed with 5 resin-bed volumes of buffer A. Polymerase was eluted from the resin with 350 mL of buffer (20 mM Tris-HCl pH 8, 50-1000 mM NaCl gradient, 2 mM EDTA, 0.5% Elugent™).

The collected protein fractions were analyzed by SDS-PAGE. Selected elution fractions were combined, slowly mixed with equal volume of 3 M (NH₄)₂SO₄ and loaded on POROS Ethyl column (23 ml volume) which was pre-equilibrated with 5 resin-bed volumes of buffer B (20 mM Tris-HCl, 1.5 M (NH₄)₂SO₄, 2 mM EDTA). The resins were washed with 5 resin-bed volumes of buffer B. Polymerase was eluted from the resin with 250 mL of buffer (20 mM Tris-HCl pH 8, 1500-0 mM (NH₄)₂SO₄ gradient, 2 mM EDTA). The collected protein fractions were analyzed by SDS-PAGE. Selected elution fractions were combined, concentrated 13 times by Amicon Ultra 15K concentrators and then dialyzed in storage buffer (20 mM Tris-HCl pH 8, 100 mM KCl, 0.1 mM EDTA, 0.5% Nonidet P40™, 0.5% Tween 20™, 50% glycerol) overnight at 4° C. After dialysis, protein sample was stored at −20° C. Protein had about 80% purity.

### Example 4. Polymerase Activity Assay

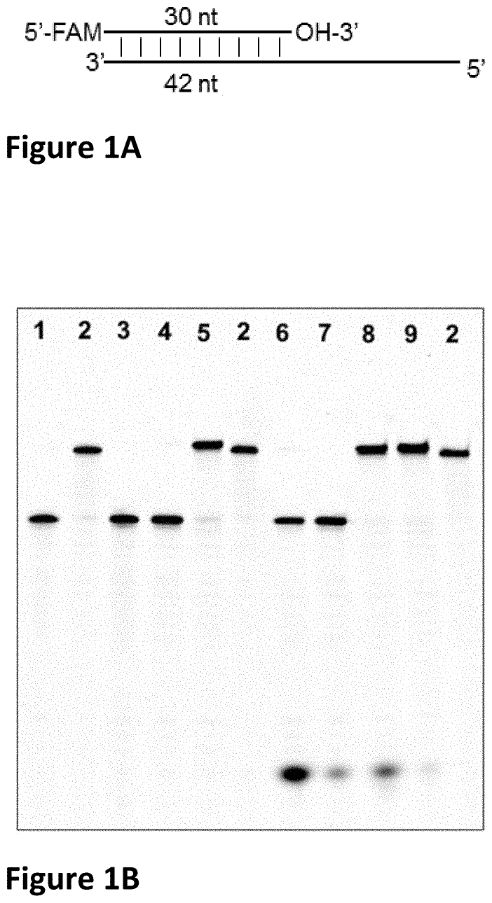

Reactions were performed in a 30 μL reaction volume containing 1× Taq Buffer with (NH₄)₂SO₄ (75 mM Tris-HCl pH 8.8 at 25° C., 20 mM (NH₄)₂SO₄, 0.01% (v/v) Tween 20), 0.2 mM dNTP, 1.6 mM MgCl₂, and 50 nM 6-fluorescein amidite (FAM)-labeled DNA-DNA oligoduplex with 12 deoxyribonucleotide 5′ overhang (hybridized SEQ ID NO: 3 and SEQ ID NO: 4, schematic structure is shown in FIG. 1A), and incubated at 60° C. for 30 min using 15U (10× concentration) and 5U (1× concentration) of Polymerase I sample (one unit of the enzyme catalyzes the incorporation of 10 nmol of deoxyribonucleotides into a polynucleotide fraction in 30 min at 60° C.). Reactions were quenched by adding 10 μL of reaction mixture to 14 μL STOP solution containing EDTA (10 mM EDTA, 98% (v/v) Formamide, 10 mg/ml Blue Dextran) and cooling the reaction down on ice. Then 10 μL of the samples denatured by heating reaction products were analyzed by denaturing urea (7 M) polyacrylamide gel (15%) electrophoresis at 50° C. (results shown in FIG. 1B).

The samples in each lane of FIG. 1B are described in Table 2.

TABLE 2
Reaction conditions for polymerase activity assay

| Sample/lane | Reaction |
|---|---|
| Number | conditions |
| 1 | 30 nucleotide control |
| 2 | 42 nucleotide control |
| 3 | DNA/DNA oligoduplex substrate (S) |
| 4 | S + Taq polymerase (Thermo Scientific ™, |
| | #EP0402) − dNTP |
| 5 | S + Taq polymerase (Thermo Scientific ™, |
| | #EP0402) + dNTP |
| 6 | S + 1X Polymerase I − dNTP |
| 7 | S + 10X Polymerase I − dNTP |
| 8 | S + 1X Polymerase I + dNTP |
| 9 | S + 10X Polymerase I + dNTP |

Samples 4 and 5 show results with reactions comprising commercially-available Taq DNA polymerase as positive control. Samples 4, 6, and 7 lacked dNTPs, and extension was not seen in any of these samples, while some exonuclease activity by Polymerase I could be observed. Sample 8 (1× Polymerase I) and sample 9 (10× Polymerase I) show extension in samples comprising dNTPs together with Polymerase I, thus showing DNA-dependent DNA polymerase activity.

### Example 5. Reverse Transcriptase Activity Assay

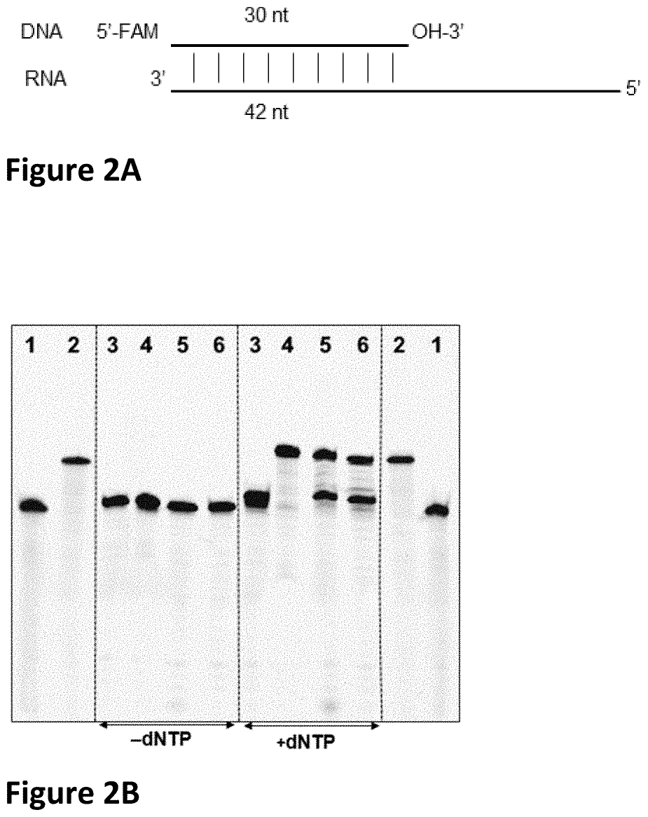

Reactions were performed in 1× Taq Buffer with (NH₄)₂SO₄ (75 mM Tris-HCl pH 8.8 at 25° C., 20 mM (NH₄)₂SO₄, 0.01% v/v Tween 20), 0.2 mM dNTP, 1.6 mM MgCl₂, and 50 nM FAM-labeled RNA/DNA oligoduplex with 12 ribonucleotide 5′ overhang (hybridized SEQ ID NO: 5 and SEQ ID NO: 4, schematic structure is shown in FIG. 2A), and incubated at 60° C. for 30 minutes using 15U and 5U of Polymerase I sample (10× and 1× concentrations, respectively). Reactions were quenched by adding 10 μL of reaction mixture to 14 μL STOP solution containing EDTA and cooling the reaction down on ice. Then, 10 μL of the samples denatured by heating reaction products were analyzed by denaturing urea (7 M) polyacrylamide gel (15%) electrophoresis at 50° C. (FIG. 2B).

The samples in each lane are described in Table 3.

TABLE 3
Reaction conditions for reverse transcriptase activity assay

| Sample/lane | Reaction |
|---|---|
| Number | conditions |
| 1 | 30 nucleotide control |
| 2 | 42 nucleotide control |
| 3 | Taq DNA polymerase (Thermo Scientific ™, #EP0402) |
| 4 | SuperScript IV RT control (Invitrogen ™, #18090010) |
| 5 | 1X Polymerase I |
| 6 | 10X Polymerase I |

In the absence of dNTPs, no extension was seen in the reaction sample, while some exonuclease activity by Polymerase I could be observed. In the samples with dNTPs, no extension was seen with the sample comprising Taq DNA polymerase, which lacks reverse transcriptase activity (sample 3+ dNTP). Sample from a reaction comprising SuperScript IV RT, a commercially-available control reverse transcriptase, showed extension (sample 4+ dNTP). As can be seen in FIG. 2B, Polymerase I shows reverse transcriptase activity—in the presence of dNTPs, it showed extension of DNA from the RNA/DNA oligoduplex substrate. This is surprising, as most of DNA-dependent DNA polymerases do not show reverse transcriptase activity, and Polymerase I does not have a sequence homology to those DNA polymerases that have such activity. Also, for reverse transcriptase activity, Polymerase I does not require other reaction buffer components or cofactors, compared to the reaction buffer used for DNA-dependent DNA polymerase activity. Additionally, as can be seen from side by side comparison of the synthesis products in lanes 8 and 9 with lane 2, an additional nucleotide is added by Polymerase I. The size of the product corresponds to the size of synthesis product by Taq DNA polymerase in lane 5, and thus denotes that Polymerase I possesses terminal transferase activity. Single nucleotide incorporation assay demonstrated Polymerase I preference to incorporate dA at 3′-end of a double-stranded template due to its terminal transferase activity (data not shown).

### Example 6. Polymerase I Primer Elongation Experiments Using Different Reaction Conditions

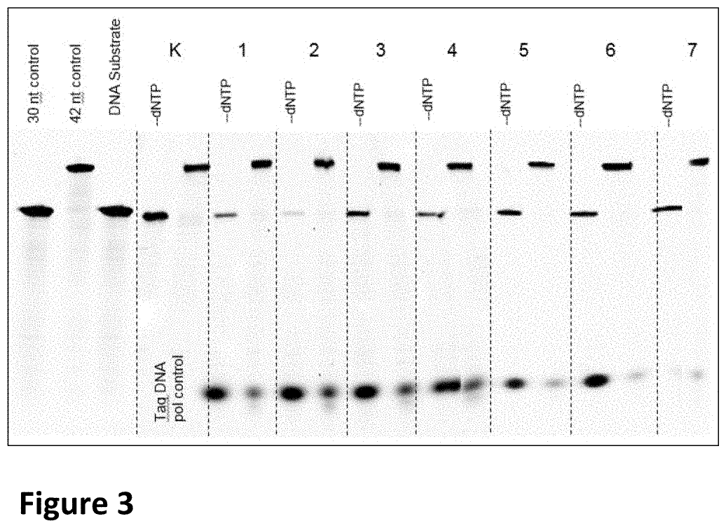

Primer elongation reactions were performed in 7 different reaction buffers (using buffer compositions listed in Table 4) and 50 nM FAM labeled DNA-DNA oligoduplex (the same as in Example 4). In each sample, 1.5U of Polymerase I was used in a total reaction volume of 30 μl. Reactions were incubated at 60° C. for 30 min. Each reaction was done in the presence of dNTPs or without dNTPs (“−dNTP” in FIG. 3). As a control, no elongation should be seen in the absence of dNTPs.

As shown in Table 4, a wide variety of buffers were tested in primer elongation experiments. For example, buffers with and without detergents and/or reducing agents were tested.

TABLE 4
Buffer compositions for primer elongation experiments

| 1x Buffer | 1 | 2 | 3 | | 4 | 5 | 6 | 7 |
|---|---|---|---|---|---|---|---|---|
| pH | 8.8 | 8.8 | 8.8 | | 8.8 | 8 | 7.9 | 7.5 |
| Tris-HCl | 75 mM | 10 mM | 20 | mM | 20 mM | 50 mM | — | 40 mM |
| Tris-Ac | — | — | — | | — | — | 33 mM | — |
| KCl | — | 50 mM | 10 | mM | 10 mM | — | 66 mM | — |
| (NH₄)₂SO₄ | 20 mM | — | 10 | mM | 10 mM | — | — | — |
| MgCl₂ | 1.6 mM | 1.6 mM | — | | — | 5 mM | — | 10 mM |
| MgSO₄ | — | — | 2 | mM | 2 mM | — | — | — |
| Mg(Ac)₂ | — | — | — | | — | — | 10 mM | — |
| DTT | — | — | — | | — | 1 mM | 1 mM | 1 mM |
| Tween 20 | 0.01% | — | — | | 0.10% | — | 0.10% | — |
| NP-40 | — | 0.08% | — | | — | — | — | — |
| Triton X100 | — | — | 0.10% | | — | | — | — |
| BSA | — | — | 0.1 | mg/mL | — | | — | — |
| dNTP | 0.2 mM | 0.2 mM | 0.25 | mM | 0.25 mM | 0.625 mM | 1.25 mM | 1.25 mM |

Results in FIG. 3 show results from reaction run in each buffer listed in Table 4 (sample number in FIG. 3 corresponds to buffer number in Table 4). Sample Kin FIG. 3 is a control sample with Taq DNA Polymerase in Buffer 1 from Table 4.

FIG. 3 shows that a wide range of buffers supported primer extension by Polymerase I in the presence of dNTPs. Primer elongation was not seen for any sample in the absence of dNTPs.

Primer elongation reactions were also performed in 10 different reaction buffers and 50 nM FAM labeled DNA-RNA oligoduplex (the same as in Example 5) to analyze reverse transcriptase activity. 7 buffers from Example 6 were used and Table 5 shows the compositions of the additional 3 buffers.

TABLE 4
Buffer compositions for reverse transcriptase experiments

| 1x Buffer | 8 | 9 | 10 | |
|---|---|---|---|---|
| pH | 9 | 7.75 | 8.3 | |
| Tris-HCl | — | — | 50 | mM |

| | | | | |
|---|---|---|---|---|
| Tris-Ac | 33 | mM | — | — |

| HEPES-KOH | — | 50 | mM | — | |
|---|---|---|---|---|---|
| KCl | — | 75 | mM | 50 | mM |

| | | | | |
|---|---|---|---|---|
| CH3COOK | 90 | mM | — | — |

| | | | | | |
|---|---|---|---|---|---|
| MgCl₂ | — | 3 | mM | 4 | mM |

| | | | | |
|---|---|---|---|---|
| Mg(Ac)₂ | 15 | mM | — | — |

| | | | | |
|---|---|---|---|---|
| DTT | — | — | 10 | mM |

| Tween 20 | 0.1% | — | | — |
|---|---|---|---|---|
| BSA | — | 0.0005 | mg/ml | — |

| | | | | | | |
|---|---|---|---|---|---|---|
| dNTP | 1.875 | mM | 0.375 | mM | 0.5 | mM |

Polymerase I demonstrated reverse transcriptase activity in all tested buffers, thus a wide range of buffer supported reverse transcriptase activity by Polymerase I in the presence of dNTPs.

Polymerase I activity in various reaction temperatures. Primer elongation reactions were performed in 1× Taq Buffer with (NH₄)₂SO₄ (75 mM Tris-HCl pH 8.8 at 25° C., 20 mM (NH₄)₂SO₄, 0.01% (v/v) Tween 20) with a FAM-labeled DNA-DNA oligoduplex substrate for 5 and 30 minutes at temperatures of 40° C., 45° C., 50° C., 55° C., 60° C., 65° C., 70° C., 75° C. and 80° C. The results (not shown) demonstrated that Polymerase I has DNA polymerase/primer extension activity at all the tested temperatures. Thus, Polymerase I is active in a wide range of temperatures. In analogous reaction with FAM-labeled RNA-DNA oligoduplex substrate (reverse transcription assay) at temperatures of 40° C., 45° C., 50° C., 55° C. and 60° C., Polymerase I reverse transcriptase activity was highest at temperatures 50° C. to 55° C. Thus, Polymerase I reverse transcriptase activity is observed at an elevated temperature.

### Example 7. Isothermal Amplification Assay

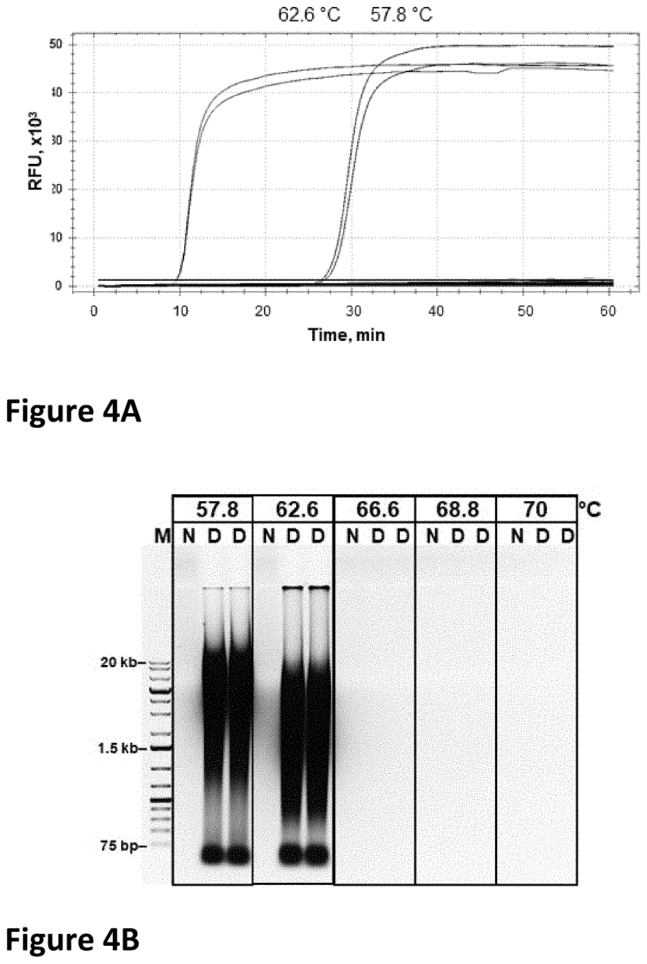

Isothermal amplification reaction was performed using circular single stranded M13mp18 DNA as a template and annealing the DNA oligonucleotide of SEQ ID NO: 6 to serve as a primer. Multiple reaction mixtures in a 1× reaction buffer (33 mM Tris-acetate (pH 7.9 at 37° C.), 10 mM Mg-acetate, 66 mM K-acetate, 0.1% (v/v) Tween 20, 1 mM DTT), comprising 6.25 M EvaGreen™, 1 mM dNTP, 1 ng/L M13mp18 ssDNA with annealed primer, 0.5 U/L of Polymerase I were prepared and incubated up to 2 hours at various temperatures in 96-well plate. Real time PCR instrument CFX96 Touch (Bio-Rad) was used for incubation and detection of fluorescence. As can be seen from FIGS. 4A and 4B, large size synthesis products accumulated, which indicates that strand displacement reaction took place. Additionally, it was confirmed that Polymerase I was able to amplify the DNA via strand displacement reaction most efficiently at temperatures from 61° C. to 63° C.

### Example 8. Evaluation of Polymerase I DNA Synthesis Fidelity

DNA substrate for Polymerase I primer extension reaction was generated by transforming E. coli bacteria with M13 phage. A single bacteria colony was selected for growing biomass for DNA extraction. Circular single stranded DNA (ssDNA) substrate was purified from growth media as single stranded M13 phage exists outside the cell. Double stranded DNA (dsDNA) as sequencing reference control was extracted from collected bacteria cells.

The ssDNA substrate for primer elongation reaction was prepared by annealing the DNA oligonucleotide M13_XbaI containing XbaI restriction endonuclease recognition sequence (SEQ ID NO: 6, GCCTGCAGGTCGACTCTAGAGGATCCCCGGGTACCGAGC, XbaI recognition sequence bolded) to the single-stranded M13mp18 DNA template. The mixture contained 2 μM primer M13_XbaI, 2 μg single stranded M13mp18 DNA, Taq DNA Polymerase reaction buffer Thermo Scientific, cat. no. B33 (with (NH₄)₂SO₄) diluted to 2× concentration, 3.2 mM MgCl₂ and nuclease free water up to 50 μl. The mixture was incubated at 99° C. for 5 min, then slowly cooled down to room temperature. M13mp18 DNA with annealed primer was linearized by digestion with 2 μl of FastDigest XbaI (Thermo Scientific, cat. no. FD0684) restriction enzyme for 60 min at 37° C. in FastDigest buffer. Double stranded M13mp18 DNA control was linearized with XbaI, accordingly.

Primer elongation (˜7 kb) reaction by Polymerase I and Taq DNA polymerases controls. Reactions were performed using 2× substrate (in 2× Taq buffer) from step 2 and adding necessary concentrations of MgCl₂, dNTPs and each polymerase. Reaction times and temperatures were optimized for every polymerase.

Polymerase I

Primer elongation reaction mix:

| | Primer extension reaction with 2x substrate from step 2 | | |
|---|---|---|---|
| | Final | | |
| Polymerase I | concentration | | component |
| 50 μl | 20 | ng/μl | 25 μl of 2X DNA substrate from step 2 |
| reaction | | | (in 2X Taq buffer with (NH₄)₂SO₄) |
| | | | (has 3.2 mM MgCl₂) |
| | 0.2 | μM | dNTP mix (Thermo Scientific ™ |
| | | | cat. No. R0192) |
| | 1.25 | U input | Polymerase I (stock 5.4 U/μl) |

H₂O up to 50 μl

Reaction mix was incubated at 60° C. for 20 minutes.

Taq DNA Polymerase

Primer elongation reaction mix:

| | Primer extension reaction with 2x substrate from step 2 | | |
|---|---|---|---|
| Taq | Final | | |
| polymerase | concentration | | component |
| 50 μl | 20 | ng/μl | 25 μl of 2X DNA substrate from step 2 |
| reaction | | | (in 2X Taq buffer with (NH₄)₂SO₄) |
| | | | (has 3.2 mM MgCl₂) |
| | 0.2 | μM | dNTP mix (Thermo Scientific ™ |
| | | | cat. No. R0192) |
| | 1.25 | U input | Taq DNA polymerase (Thermo |
| | | | Scientific ™ cat. No. EP0402) |

H₂O up to 50 μl

Reaction mix was incubated at 72° C. for 90 minutes.

˜7 kb linear DNA products from step 3 were purified with Thermo Scientific™ GeneJET PCR Purification Kit (cat. no. K0701). Purified product was treated with RecJ_{f} exonuclease (NEB cat. no. M0264S) in 1× NEBuffer 2 to hydrolyse single stranded DNA ends (10 U to 1 μg of DNA product) if the primer was not extended completely. After this step DNR product was purified with Thermo Scientific™ GeneJET PCR Purification Kit (cat. no. K0701).

UMI tagmentation to incorporate barcodes using randomized transposon ends was used to detect rare mutations by reducing sequencing background as described in U.S. 62/899,468. Transposome complexes comprising transposon end nucleic acids with 12 randomized positions (comprising transferred (top) strand SEQ ID NO: 7 and non-transferred (bottom) strand SEQ ID NO: 8) were used, accordingly. 25 ng of each purified DNA product was premixed with 2 μl of MuA complex in 1× Fragmentation Reaction Buffer (Thermo Scientific™ MuSeek™ Library Preparation Kit, Illumina™ compatible, Cat. No. K1361) in separate vials. Fragmentation was carried out in 30 μl reactions for 5 minutes at 30° C., then stopped by adding 3 μl of 4.4% SDS solution.

Fragmented DNA was subjected to size selection using the Collibri™ DNA Library Cleanup Kit (Invitrogen, Cat. No. A38584096). The sample was mixed with 50 μl of magnetic beads and incubated for 5 min at room temperature. After a short spin, tubes were placed in a magnetic rack until the solutions were cleared. The supernatant was aspirated carefully without disturbing the beads and discarded. The beads were resuspended in 102 μl of elution buffer and placed back into magnetic rack until the solutions were cleared. 100 μl of supernatant was transferred in a new tube, mixed with 60 μl of magnetic beads, and incubated for 5 min at room temperature. After a short spin, the tubes were placed in a magnetic rack until the solutions were cleared. Supernatant was transferred in a new tube, mixed with 25 μl of magnetic beads, and incubated for 5 min at room temperature. After a short spin, the tubes were placed in a magnetic rack until the solutions were cleared. The supernatant was aspirated carefully without disturbing the beads and discarded. The beads were washed twice by incubating for 30 seconds with 200 μL 85% ethanol followed by removing the supernatant after 30 seconds of incubation. The tubes were given a short spin to collect excess ethanol and placed back into magnetic rack. Excess ethanol was removed, the beads were then air-dried by opening the tube caps for two minutes, allowing the remaining ethanol to evaporate. The tubes were removed from the magnetic rack, the beads were resuspended in 25 μL of elution buffer (10 mM Tris-HCl (pH 8.3)) and placed back into magnetic rack. DNA was eluted by carefully aspirating the supernatant.

Primers were designed to anneal to the transposon end nucleic acid sequence directly upstream of the N12 randomized sequence. Fragmented DNA containing random sequences was subjected to PCR amplification using Collibri Library Amplification Master Mix (Invitrogen, Cat. No. A38539050) to introduce Illumina P5/P7 adapters and library barcodes using the following primers:

P5-D501 (SEQ ID NO: 9):
AATGATACGGCGACCACCGAGATCTACACTATAGCCTATGCG
ACACTCGTGAAACGCTTTCGCGTTT
P5-D502 (SEQ ID NO: 10):
AATGATACGGCGACCACCGAGATCTACACATAGAGG
CATGCGACACTCGTGAAACGCTTTCGCGTTT
P5-D503(SEQ ID NO: 11):
AATGATACGGCGACCACCGAGATCTACACCCTATCCTATGCG
ACACTCGTGAAACGCTTTCGCGTTT
P7-D701(SEQ ID NO: 12):
CAAGCAGAAGACGGCATACGAGATATTACTCGCGAGGTCGAGT
GCATGAAACGCTTTCGCGTTT
P7-D702(SEQ ID NO: 13):
CAAGCAGAAGACGGCATACGAGATTCCGGAGACGAGGTCGAGTG
CATGAAACGCTTTCGCGTTT
P7-D703(SEQ ID NO: 14):
CAAGCAGAAGACGGCATACGAGATCGCTCATTCGAGGTCGA
GTGCATGAAACGCTTTCGCGTTT

A minimal amount of template (0.1 μL) was taken for amplification. The cycling protocol was: 1 cycle for 3 min at 66° C.; 1 cycle for 30 sec at 98° C.; 20 cycles for 15 sec at 98° C.; 30 sec at 60° C.; 30 sec at 72° C.; 1 cycle for 1 min at 72° C. Amplified library was purified from reaction mixture using the Collibri™ DNA Library Cleanup Kit. (Invitrogen, Cat. No. A38584096). 50 μL of PCR reaction was mixed with 40 μL of magnetic beads and incubated for 5 min at room temperature. After a short spin, the tubes were placed in a magnetic rack until the solutions were cleared. The supernatant was aspirated carefully without disturbing the beads and discarded. The beads were resuspended in 50 L of elution buffer (10 mM Tris-HCl (pH 8.3) and mixed with 50 μL of fresh magnetic beads. After a short spin and incubation for 5 min at room temperature, the tubes were placed in a magnetic rack until the solutions were cleared. The supernatant was aspirated carefully without disturbing the beads and discarded. The beads were washed twice by incubating for 30 seconds with 200 μL 85% ethanol and removing the supernatant after 30 seconds of incubation. The tubes were given a short spin to collect excess ethanol and placed back into magnetic rack. Excess ethanol was removed, the beads were then air-dried by opening the tube caps for two minutes, allowing remaining ethanol to evaporate. The tubes were removed from the magnetic rack, the beads were resuspended in 22 μL of elution buffer (10 mM Tris-HCl (pH 8.3)) and placed back into magnetic rack. DNA was eluted by carefully aspiring the supernatant. Agilent analysis (Agilent High Sensitivity DNA Kit) and qPCR using Collibri Library Quantification Kit (Invitrogen, Cat. No. A38524500) were performed for library quality assessment. Libraries were pooled and sequenced on MiSeq instrument in paired 150 bp mode using custom primers:

Read 1: (SEQ ID NO: 15):
ATGCGACACTCGTTCGTGCGTCAGTTCA
Read 2: (SEQ ID NO: 16):
CGAGGTCGAGTGCAGTTCGTGCGTCAGTTCA
Index read: (SEQ ID NO: 17):
TGAACTGACGCACGAACTGCACTCGACCTCG

Generated sequencing data were analyzed by grouping reads to barcode (UMI) families and then calling polymerase errors. First, barcode sequences were extracted from reads using UMI-tools (v0.5.3). Next, adapters and low-quality sequences were trimmed using BBMAP (v37.17). Resulting reads were aligned with BWA aligner (v0.7.15) and grouped to families using UMI-tools group adjacency algorithm with hamming distance 1 (v0.5.3). Polymerase errors were called only if they are present in all reads in the UMI family, otherwise they were discarded as sequencing error.

According to fidelity measurement results, the error rate of Polymerase I was determined to be 4×10⁻⁵, whereas the error rate of Taq DNA polymerase was 9×10⁻⁵. Thus, Polymerase I fidelity is more than 2 times higher compared to Taq DNA polymerase, i.e. the error rate is more than 2-times lower than that of Taq DNA polymerase.

Fidelity of several other DNA polymerases such as Bst DNA polymerase, phi29 DNA polymerase will be determined using the same conditions.

### Example 9. Evaluation of cDNA Synthesis Fidelity of Polymerase I

Fidelity of a polymerase can be measured in a number of ways. For Polymerase I, the fidelity could be evaluated using NGS sequencing after cDNA synthesis.

First, a yeast Komagataella phaffii 76273 RNA library will be constructed using Invitrogen Collibri Stranded RNA Library Prep Kit for Illumina (www.thermofisher.com/uk/en/home/life-science/sequencing/next-generation-sequencing/ngs-library-preparation-illumina-systems/collibri-stranded-rna-library-prep-kits.html). The library will be prepared following the workflow provided in the Kit description except that the reverse transcription reaction will be performed using Polymerase I instead of the SuperScript IV Enzyme Mix. Then, the library will be sequenced on Illumina MiSeq NGS sequencing platform. The sequence of the library prepared by Polymerase I will be compared to the expected sequence for the library and the number of errors may be determined. The number of errors divided by the length of each sequence in the library produces an error rate and the error rate for each individual member of the library may be averaged to determine a population error rate. Seeing the error rate for each individual member of the library also provides useful information on the consistency of the Polymerase I. The fidelity may be determined as 1/error rate.

### Example 10. Variants of Polymerase I

Polymerase I domain structure was analyzed using Interproscan (https://www.ebi.ac.uk/interpro/search/sequence/) and Pfam-based protein signature database. Analysis identified 5′-exonuclease domain in N-terminal part of Polymerase I, up to and including 283rd amino acid residue of SEQ ID NO: 1. Polymerase I having reduced or lacking 5′-exonuclease domain (for example, inactivated by a mutation or by deletion of a part or of a full 5′-exonuclease domain) would retain polymerase activity. Such polymerase would have an improved strand-displacement activity compared to full length Polymerase I and would be useful in various isothermal amplification methods, such as multiple displacement amplification (MDA) or loop-mediated isothermal amplification (LAMP). Variants of Polymerase I that are useful in such applications include polymerase variants derived from SEQ ID NO: 1 by deletion of a consecutive amino acid sequence from the 1st to any amino acid up to 283rd amino acid residue. Examples of deletions of consecutive amino acid sequences to provide a variant Polymerase I as described herein may be deletion of a consecutive amino acid sequence from the 1st to any amino acid up to and including amino acid position 8, 19, 48, 58, 84, 111, 135, 160, 169 or 283 of SEQ ID NO: 1.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. (canceled)

2. A method of in vitro nucleic acid synthesis comprising contacting at least one primer and at least one template nucleic acid with a DNA polymerase in the presence of at least one dNTP in a buffer and incubating the reaction, wherein a DNA polymerase comprises:
(1) an amino acid sequence having at least 70%, 75%, 80%, 85%, 90%, 95%, 96%, 97%, 98%, or 99% identity to SEQ ID NO: 1, or
(2) an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue.

3. The method of claim 2, wherein the template nucleic acid is RNA.

4. The method of claim 2, wherein the template nucleic acid is DNA.

5. A method of reverse transcription and DNA amplification comprising contacting at least one primer and at least one template RNA with a DNA polymerase in the presence of at least one dNTP and incubating the reaction in a buffer under conditions for reverse transcription followed by conditions for DNA amplification, wherein a DNA polymerase comprises:
(1) an amino acid sequence having at least 70%, 75%, 80%, 85%, 90%, 95%, 96%, 97%, 98%, or 99% identity to SEQ ID NO: 1, or
(2) an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue.

6. The method of claim 2, wherein the DNA polymerase comprises SEQ ID NO: 1.

7. The method of claim 2, wherein the DNA polymerase comprises amino acid sequence derived from SEQ ID NO: 1 by deletion of a consecutive amino acid sequence from the 1st to any amino acid up to 283rd amino acid residue.

8. The method of claim 7, wherein the DNA polymerase comprises amino acid residues 284-878 of SEQ ID NO: 1.

9. (canceled)

10. The method of claim 2, wherein the incubating is performed at temperature from 40° C. to 80° C.

11. The method of claim 10, wherein the incubating is performed at 40° C. or higher.

12. The method of claim 11, wherein the incubating is performed at 60° C. or higher.

13. An isolated DNA polymerase comprising
(1) an amino acid sequence having at least 70%, 75%, 80%, 85%, 90%, 95%, 96%, 97%, 98%, or 99% identity to SEQ ID NO: 1, or
(2) an amino acid sequence derived from amino acid sequence having at least 70%, 75%, 80%, 85%, 90%, 95%, 96%, 97%, 98%, or 99% identity to SEQ ID NO: 1 by deletion of a consecutive amino acid sequence from the 1st to any amino acid up to 283rd amino acid residue.

14. The DNA polymerase of claim 13 comprising SEQ ID NO: 1.

15. The DNA polymerase of claim 13 comprising amino acid sequence derived from SEQ ID NO: 1 by deletion of a consecutive amino acid sequence from the 1st to any amino acid up to 283rd amino acid residue.

16. The DNA polymerase of claim 15 comprising amino acid residues 284-878 of SEQ ID NO: 1.

17. (canceled)

18. A composition comprising the DNA polymerase of claim 13 and a storage solution.

19. A composition comprising the DNA polymerase of claim 13 and a buffer.

20. A kit comprising the DNA polymerase of claim 13 and at least one buffer suitable for use in a polymerase reaction.

21. An isolated nucleic acid encoding the DNA polymerase of claim 13.

22. A vector comprising the nucleic acid of claim 21.

23. A cell comprising the vector of claim 22.

24-25. (canceled)
